(19) United States
(12) Patent Application Publication
(10) Pub. No.: US 2022/0412994 A1
(43) Pub. Date: Dec. 29, 2022

(54) BIOMARKER OF DRUG-INDUCED CELLULAR TOXICITY AND DEPRESSION

(71) Applicant: Randox Laboratories Ltd, Crumlin Antrim (GB)

(72) Inventors: Paul Innocenzi, Crumlin Antrim (GB); Mark Ruddock, Crumlin Antrim (GB); John Lamont, Crumlin Antrim (GB); Peter Fitzgerald, Crumlin Autrim (GB)

(21) Appl. No.: 17/775,217

(22) PCT Filed: Nov. 6, 2020
(86) PCT No.: PCT/EP2020/081273

§ 371 Date: May 6, 2022

(30) Foreign Application Priority Data
Nov. 7, 2019 (GB) 1916185.0

(51) Int. Cl. G01N 33/68

(52) U.S. Cl. CPC G01N 33/6896; G01N 2800/304; G01N 2800/52

(57) ABSTRACT

Described is the use of GFAP as a marker of drug-induced cellular toxicity and depression.

### BACKGROUND

Post-traumatic stress disorder (PTSD) is a syndrome resulting from exposure to actual or threatened serious injury, death or sexual assault (1). PTSD affects civilians and especially active duty military personnel. Risk factors include gender, prior traumatic exposure, pre-existing mental illness, lower socio-economic status, lower intelligence and childhood adversity. Post-traumatic factors include development of acute stress disorder (ASD), other stresses such as financial problems, subsequent adverse life events and lack of social support. Psychiatric conditions such as PTSD are poorly understood and there is a wide heterogeneity in how the illness manifests in individuals. Following a traumatic event there may be feelings of anxiety, sadness, stress, nightmares, intrusive memories about the event and problems sleeping. However, these symptoms do not necessarily mean that an individual has PTSD. Diagnostic and Statistical Manual of Mental Disorders (DSM-DSM-5 is the latest version) is used by clinicians and psychiatrists to diagnose psychiatric illness. The DSM describes symptoms and provides statistics including gender, age of onset and effect of treatment. The main issue with the DSM is validity. A statement issued by the National Institute for Mental Health (NIMH) stated that ‘the DSM-5 represented the best information currently available for clinical diagnosis of mental disorders.’ A diagnosis is valid when it accurately describes a patient's condition or disorder. However, the diagnoses described in the DSM-5 are not objectively described physical medical conditions like heart disease, diabetes, cancer etc., but are symptoms and behaviours reported by patients and interpreted by the clinicians. Possible risks of such an approach include misdiagnosis or over-diagnosis. The clinician's tendency to look for, find, and interpret the results, may lead to confirmation bias. As such, patients can potentially be labeled as having a disorder simply because their behaviour does not always conform to the current ‘ideal’. To date, no clinically validated biological biomarker or biomarker combination (notably protein biomarkers) has been found to assist in the diagnosis, treatment and management of patients with PTSD. Symptoms and comorbidity with other neuropsychiatric disorders e.g. depression and anxiety, suggest that a single biomarker is unlikely to be diagnostic. A key co-morbidity of PTSD, depression, represents a greater societal challenge than PTSD itself, and it is currently estimated that on average 10% of individuals will be subject to depression during their life-time. Diagnosing depression is equally difficult and requires an in-depth personal history and clinical examination that seeks to identify key changes in emotions, memory, physical ailments, personality etc. The subjective process can lead to up to a 50% misdiagnosis rate which ultimately affects patient care. A more objective approach to the diagnosis of depression is an ongoing medical challenge and protein biomarkers of depression is an active research area.

A study was developed to investigate whether a biomarker or combination of biomarkers, with clinical risk factors, could be used to aid clinicians identify and/or stratify patients ‘at risk’ of PTSD. Unexpectedly, the study identified a biomarker of depression and drug-induced cellular damage.

### SUMMARY OF THE INVENTION

During an investigation of possible blood biomarkers of post-traumatic stress disorder, it was surprisingly found that glial fibrillary acidic protein (GFAP) was upregulated in individuals with PTSD and non-PTSD cohorts who were taking medication.

The invention describes an ex vivo method of assessing the cellular effect of a drug, to which an individual has previously been exposed or which has been added to an in vitro cell line, by measuring the amount of GFAP in an in vitro sample of the individual or in the in vitro cell line and in which an increase in the amount of measured GFAP is indicative of cellular toxicity or damage.

From the same patient cohorts, it was further identified that depressed individuals had higher amounts of blood GFAP than non-depressed individuals.

The use of GFAP as a biomarker of drug-induced cellular toxicity and depression could support its use in clinical medicine, clinical trials and in precision medicine as a companion diagnostic for identifying potentially toxic drugs. The use of GFAP as a psychiatric disease biomarker could provide greater objectivity and accuracy for the clinical diagnosis of depression.

### FIGURES

FIG. 1 Mean GFAP levels in individuals from PTSD cohort (three left bar diagrams) and non-PTSD cohort (three right bar diagrams). As the GFAP assay has a cut-off value of 0.16 ng/ml and a number of individuals in the healthy cohort had no detectable GFAP for the statistical analyses these individuals were assigned the cut-off value; hence values depicted in the Healthy cohort will be an overestimate.

FIG. 2 Mean GFAP levels for individuals in the PTSD cohort taking prescribed SSRI medication and prescribed non-SSRI medication

FIG. 3 Mean GFAP levels for individuals with depression taking prescribed SSRI medication and prescribed non-SSRI medication (upper) and without with depression taking prescribed SSRI medication and prescribed non-SSRI medication (lower)

FIG. 4 Mean GFAP levels for individuals in the PTSD cohort taking prescribed SSRI medication who were depressed and not depressed (upper) and taking prescribed non-SSRI medication who were depressed and not depressed (lower)

FIG. 5 Follow-up study. Mean GFAP levels for individuals not taking prescribed medication, individuals taking prescribed medication which does not act upon brain cell receptors or brain physiological pathways (non-brain impacting medication) and individuals taking prescribed medication which do (brain impacting medication).

### DETAILED DESCRIPTION OF THE INVENTION

A study, comprising a PTSD cohort and non-PTSD cohort, whose aim was to detect blood-based biomarkers in individuals to support PTSD diagnosis unexpectedly identified a biomarker of drug-induced cellular damage and depression. PTSD individuals suffer from, and are partly defined by, a group of co-morbid conditions including depression and anxiety-related disorders, and are often prescribed several medications to counter the conditions. The PTSD study assessed several biomarker levels but is was the noticeable variation in levels of GFAP that provoked a further analysis.

GFAP is a structural protein found mainly in brain astrocytes and is a peripheral blood biomarker of stroke and traumatic brain damage (TBI) (WO2018096049, WO2018095872), conditions that may be a risk factor for dementia. GFAP exists in one or more of the three characterized GFAP full-length isoforms listed in the Uniprot database, GFAP isoform 1 of Uniprot number P14136-1 synonym GFAP alpha, GFAP isoform 2 of Uniprot number P14136-2 synonym GFAP delta, GFAP isoform 3 of Uniprot number P14136-3 synonym GFAP epsilon, each with or without post-translational modification (PTM). ‘GFAP species’, means any GFAP-based protein including all native GFAP isoforms with and without PTM and other GFAP isoforms such as GFAP beta, GFAP kappa, GFAP gamma, GFAPΔEx6, GFAPΔ135, GFAPΔ164 and GFAPΔEx7 (Moeton et al 2016), GFAP multimers (dimers, tetramers etc based on any of the various isoforms), GFAP fragments and peptides structurally unique to GFAP. A ‘GFAP fragment’ (breakdown product) is derived from enzymatically digested native GFAP; a GFAP breakdown product could also comprise a PTM. When used herein, the term ‘GFAP’ corresponds to any GFAP species and any GFAP breakdown product, unless otherwise noted or contextually inferred (see WO2018096049 for detailed definition of GFAP species and breakdown products). The brain is composed of two major cell types, neurons and the more abundant glial cells, the latter being composed of oligodendrocytes and astrocytes. When an individual is subject to stroke or TBI, brain cell death results and the cytosolic content of the cells is released into the extracellular milieu and can make its way into systemic circulation. As part of the release, GFAP can enter systemic circulation, and a blood test to identify GFAP together with patient history can confirm the condition, and whether a stroke or TBI has occurred, highlighting cell death and brain damage. GFAP is essentially undetected in the blood of a healthy individual (Mayer 2013).

The further analysis compared GFAP levels in i. a PTSD cohort and ii. a non-PTSD cohort (the control group) in the following categories: i. PTSD depressed patients on medication vs patients on medication vs PTSD patients neither depressed nor taking medication and ii. depressed patients on medication vs patients on medication and not depressed vs healthy patients (not taking medication and disease-free). It was surprisingly found that in both the PTSD cohort and the non-PTSD cohort, the level of GFAP in the blood of the medicated and non-medicated groups were significantly different; in both cases GFAP levels were higher in the medicated groups.

A first aspect of the invention is a method of assessing the cellular toxicity of a drug comprising measuring the amount of GFAP in an in vitro sample taken from an individual who has previously been exposed to the drug or in an in vitro cell line or cell model that has been exposed to the drug and comparing the amount of GFAP measured to a control measurement in which a GFAP measurement taken from the in vitro sample or in vitro cell line or cell model which is greater than the control measurement is indicative of cellular toxicity.

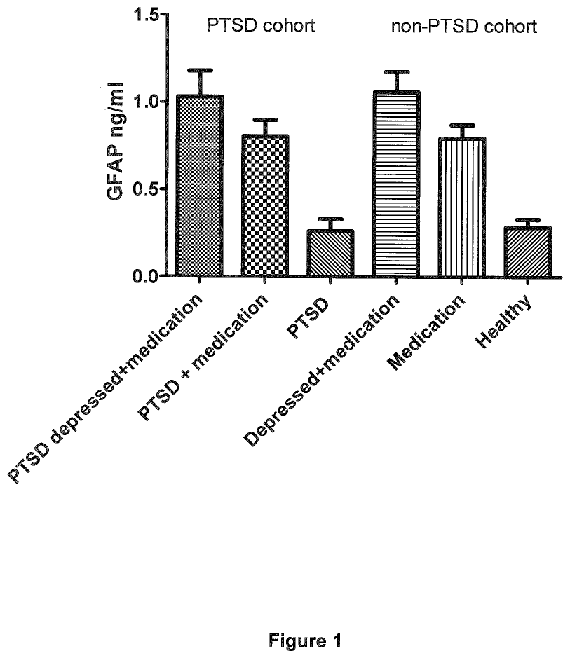

Further analysis highlighted that there was no difference between GFAP levels in unmedicated PTSD patients without co-morbidities and healthy non-PTSD individuals (see Results and FIG. 1). However, depression in an individual in both the PTSD and non-PTSD cohorts resulted in a small but significant increase in GFAP levels (see Results and FIG. 1).

A further aspect of the invention is a method of supporting a diagnosis of depression in an individual comprising measuring the amount of GFAP in an in vitro sample of the individual, and comparing the amount of GFAP measured to a control measurement in which a GFAP measurement taken from the sample which is greater than the control measurement is supportive of the individual being depressed.

In the context of the present invention, a “control measurement” or “control value” or “control level” is understood to be the level of GFAP typically found in healthy individuals. The control level of a biomarker may be determined by analysis of a sample isolated from a healthy individual or may be the level of the biomarker understood by the skilled person to be typical for a healthy individual. The control value may be a range of values considered by the skilled person to be a normal level for the biomarker in a healthy individual. The skilled person would appreciate that control values for a biomarker may be calculated by the user analysing the level of the biomarker in a sample from a healthy individual or by reference to typical values provided by the manufacturer of the assay used to determine the level of the biomarker in the sample. Alternatively, the control value is a value taken from an in vitro sample of the same individual when classified as healthy. The control measurement can be a threshold amount (also known as a cut-off value) or absolute amount of a suitable GFAP species or GFAP breakdown product. The word “individual” in the context of the current invention applies to any mammal, but is preferably homo sapiens.

In a preferred embodiment, in relation to patient drug therapy or patient depression diagnosis, the control measurement is preferably a measured amount of GFAP in an in vitro sample taken from the individual prior to drug therapy or prior to the onset of depression. The phrase “prior to” implies at any time point before the event described. During the Pharma drug development phase and prior to clinical trials, potential drug toxicity can be assessed by administering the drug to a mammal other than homo sapiens or by administering/exposing to an in vitro cell line or an in vitro cell model a drug (which includes cells extracted from healthy or diseased animals/patients) and comparing the GFAP levels in an ex vivo sample from the mammal or from the cellular milieu before and after addition of drug. The terms ‘cellular toxicity’ and ‘cellular damage’ are used synonymously herein. Thus a preferred embodiment of the invention is a method of assessing the cellular toxicity of a drug comprising measuring the amount of GFAP in an in vitro sample taken from the individual or in the in vitro cell line or cell model at a point after the an individual an in vitro cell line or cell model has been exposed to the drug, and comparing the amount of GFAP measured to a control measurement; in which the control measurement used to compare the GFAP measured in the in vitro sample taken from the individual, in the in vitro cell line or cell model, is a GFAP measurement taken from an in vitro sample of the individual, the in vitro cell line or cell model prior to exposure to the drug, and in which a GFAP measurement which is greater than the control measurement is indicative of cellular toxicity; the control measurement or value which is used to compare the amount of GFAP in the in vitro cell line or cell model following drug addition to the in vitro cell line or cell model can be a stored database control measurement or, preferably, the amount of GFAP measured in the in vitro cell line or cell model prior to addition of drug. Therefore, in a further preferred embodiment of the method of the invention is an assessment of the cellular toxicity of a drug comprising measuring the amount of GFAP in an in vitro cell line or cell model (the control measurement) then exposing a drug to the in vitro cell line or cell model, further measuring the GFAP amount in the in vitro sample or in vitro cell line or cell model, in which an amount of GFAP which is greater in the further measurement than the control measurement indicates cellular toxicity. Exposure of the individual, cell line or cell model to the drug, the time point of ingestion, addition or application of the drug, can take place hours, days, weeks or months prior to implementing the methods of the invention to analyze GFAP concentration in in vitro samples, cell lines or cell models.

The term “cell model” is any group of cells that is not a recognized cell line, including cells taken from an animal or individual. A group of cells includes a traditional cell culture and cells organized at the organoid and tissue level. The “amount” of a biomarker refers to the quantity, expression level or concentration of the biomarker within the sample. The amount of a biomarker may also refer to the biomarker measurement expressed as a ratio or percentage of the amount of one or more other analytes. The amount of one or more such other analytes may remain consistent in most samples or conditions. By way of example, the other analytes could be albumin, 13-actin or total matrix protein. The amount of a biomarker may also refer to the biomarker measurement expressed as a ratio or percentage of the amount of one or more other analytes, where the amount of the one or more other analytes is proposed to hold some biochemical significance to the clinical condition of interest. The purported mechanism of action of the active ingredients in the majority of drugs (therapeutic drugs) used in the study include molecules acting upon one or more cell receptor/neurotransmitter-impacting systems located in the brain such as serotonin transporter molecules, norepinephrine transporter molecules, dopamine transporter molecules, serotonin receptor agonist/antagonists (interacting with the ‘5-HT’ receptor family), dopamine receptor agonist/antagonists (interacting with the ‘DT’ receptor family), adrenergic receptor agonist/antagonists (interacting with the ‘alpha’ and ‘beta’ receptor families, including beta-2-adrenergic receptor agonists), GABA receptor agonist/antagonists (interacting with the ‘GABA_{A}’ and ‘GABA_{B}’ receptor families), acetylcholine receptor agonist/antagonists (interacting with the ‘muscarinic’ and ‘nicotinic’ receptor families), glutamate receptor agonists/antagonists (e.g. NMDA receptor agonist/antagonists), opioid receptor agonist/antagonists (interacting with the ‘delta’, ‘kappa’, ‘mu’, ‘nociceptin’ receptor families), histamine receptor agonist/antagonists (the ‘H₃’ receptor). Other prescribed medications include ACE inhibitors, anti-bacterials and HNG-CoA reductase inhibitors (statins). Reference to ‘medication’ herein implies prescribed medication unless otherwise qualified. The mechanism of action and other pharmacodynamic properties of the various therapeutic drugs mentioned herein can be consulted in standard pharmacopoeias.

In a preferred embodiment, the drug to which the individual has been exposed is preferably one that interacts with brain-located cellular receptors (membranous and cytosolic) or one that interacts with physiological pathways in the brain (action on a transporter), or a drug targeted to an organ other than the brain that interacts with brain-located cellular receptors or impacts/interferes physiological pathways of the brain. Alternatively, the drug is for use in treating a brain-related pathology; the brain-related pathology may include depression, anxiety, stress, panic disorder, pain, epilepsy, dementia, suicidal ideation, Alzheimer's disease, Parkinson's disease. The drug may include a selective-serotonin reuptake inhibitor (SSRI), a selective norepinephrine reuptake inhibitor (SNRI), a selective dopamine reuptake inhibitor (SDRI), serotonin receptor agonist/antagonists (interacting with the ‘5-HT’ receptor family), dopamine receptor agonist/antagonists (interacting with the ‘DT’ receptor family), adrenergic receptor agonist/antagonists (interacting with the ‘alpha’ and ‘beta’ receptor families, including beta-2-adrenergic receptor agonists), GABA receptor agonist/antagonists (interacting with the ‘GABA_{A}’ and ‘GABA_{B}’ receptor families), acetylcholine receptor agonist/antagonists (interacting with the ‘muscarinic’ and ‘nicotinic’ receptor families), glutamate receptor agonists/antagonists (e.g. NMDA receptor agonist/antagonists), opioid receptor agonist/antagonists (interacting with the ‘delta’, ‘kappa’, ‘mu’, ‘nociceptin’ receptor families), histamine receptor agonist/antagonists (the ‘H₃’ receptor); or a drug targeting an organ other than the brain that interacts with brain-located cellular receptors or indirectly disrupts brain physiological pathways. Preferably the drug results in a change in homeostasis to neurotransmitter-related physiological pathways. The medication (drugs) prescribed to individuals involved in the study are listed in the Methods and Results section. In a preferred embodiment of the invention the drug whose toxicity is being assessed is a drug intended for use in or is used in treating a neuropsychiatric condition; the condition is preferably depression, anxiety, panic disorder, suicidal ideation or stress. The condition is most preferably depression. The drug can be any drug tested for use in or used to treat a neuropsychiatric condition but is preferably a neurotransmitter reuptake inhibitor such as an SSRI, SNRI, or SDRI or a neurotransmitter receptor agonist or antagonist (includes partial agonists/anatagonists), especially a serotonin or dopamine receptor; GFAP levels were found in the study to be particularly affected by drugs of the neurotransmitter reuptake inhibitor class.

Preferably the in vitro biological sample analyzed is a blood, plasma or serum sample, but may also be cerebrospinal fluid (CSF), urine or saliva. The determination of the level of a biomarker may be done on one or more samples of the patient. The sample may be obtained from the patient by methods routinely used in the art.

A further aspect of the invention is a method of treatment of an individual with a brain-related condition in which a drug is prescribed to the individual and after ingestion of the drug a sample is taken from the individual and the amount of GFAP in the sample is measured; if the amount of GFAP is greater than a control value a decision is made as to whether to replace the drug with a different drug or to supplement the drug with medication which counteracts the drugs side-effect. The control value can be any value of GFAP that has been medically acknowledged to be a normal or healthy value for the individual i.e. a value not considered to imply a brain-related disease or condition. Preferably, the control value is a level of GFAP that has been measured in the individual at a time point prior to the administration of the prescribed drug.

The invention also describes a method of treatment of a mammal or an in vitro cell line or cell model with a drug that binds to receptors located in the brain of a mammal or one that interacts with physiological pathways in the mammalian brain in which the drug is administered to the mammal or the in vitro cell line or cell model and following administration of the drug the level of GFAP is measured in an ex vivo sample taken from the mammal or in the cellular milieu of the cell line or cell model and the level of GFAP measured is compared to a control value. The control value can take on the values described previously.

There are many analytical techniques that can be used to measure GFAP, but the preferred way of the current invention, and common to most clinical laboratories and clinical tests, is by way of an immuno-based or antibody-based test. Such tests can include competitive assay formats, immunoturbidimetric assay formats and sandwich assay formats using substrates such as slides, chips, beads, microtitre plates etc., which may contain hydrophobic or hydrophilic coatings and may be chemically-activated to enable binding or capture agents to be attached. The term “antibody” refers to an immunoglobulin which specifically recognises an epitope on a target as determined by the binding characteristics of the immunoglobulin variable domains of the heavy and light chains (VHS and VLS), more specifically the complementarity-determining regions (CDRs). Many potential antibody forms are known in the art, and in the context of the current invention may include, but are not limited to, a plurality of intact monoclonal antibodies or polyclonal mixtures comprising intact monoclonal antibodies, antibody fragments (for example Fab, Fab′, and Fv fragments, linear antibodies single chain antibodies and multispecific antibodies comprising antibody fragments), single-chain variable fragments (scFvS), multi-specific antibodies, chimeric antibodies, humanized antibodies and fusion proteins comprising the domains necessary for the recognition of a given epitope on a target. Antibodies may also be conjugated to various detectable labels to enable detection, including but not limited to radionuclides, fluorophores, dyes or enzymes including, for example, horseradish peroxidase, biotin and alkaline phosphatase. The term “binds specifically”, in the context of antibody-epitope interactions, refers to an interaction wherein the antibody and epitope associate more frequently or rapidly, or with greater duration or affinity, or with any combination of the above, than when either antibody or epitope is substituted for an alternative substance, for example an unrelated protein. Generally, but not necessarily, reference to binding means specific recognition. Techniques known in the art for determining the specific binding of a target by an antibody or lack thereof include but are not limited to, FACS analysis, immunocytochemical staining, immunohistochemistry, western blotting/dot blotting, ELISA, affinity chromatography. By way of example and not limitation, specific binding, or lack thereof, may be determined by comparative analysis with a control comprising the use of an antibody which is known in the art to specifically recognize said target and/or a control comprising the absence of, or minimal, specific recognition of said target (for example wherein the control comprises the use of a non-specific antibody). Said comparative analysis may be either qualitative or quantitative. It is understood, however, that an antibody or binding moiety which demonstrates exclusive specific recognition of a given target is said to have higher specificity for said target when compared with an antibody which, for example, specifically recognises both the target and a homologous protein.

Accuracy of a diagnostic method is best described by its receiver-operating characteristics (ROC) (Zweig, M. H., and Campbell, G., Clin. Chem. 39 (1993) 561-577). The ROC graph is a plot of all of the sensitivity/specificity pairs resulting from continuously varying the decision threshold over the entire range of data observed. A ROC plot depicts the overlap between the two distributions by plotting the sensitivity versus 1—specificity for the complete range of decision thresholds. On the y-axis is sensitivity, or the true-positive fraction defined as [(number of true-positive test results)/(number of true-positive+number of false-negative test results)]. This has also been referred to as positivity in the presence of a disease or condition. It is calculated solely from the affected subgroup. On the x-axis is the false-positive fraction, or 1—specificity [defined as (number of false-positive results)/(number of true-negative+number of false positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/specificity pair corresponding to a decision threshold. A test with perfect discrimination (no overlap in the two distributions of results) has an ROC plot that passes through the upper left corner, where the true-positive fraction is 1.0 or 100% (perfect sensitivity), and the false-positive fraction is 0 (perfect specificity). The theoretical plot for a test with no discrimination (identical distributions of results for the two groups) is a 45° diagonal line from the lower-left corner to the upper right corner. Most plots fall in between these two extremes. Qualitatively, the closer the plot is to the upper left corner, the higher the overall accuracy of the test. One convenient way to quantify the diagnostic accuracy of a laboratory test is to express its performance by a single number. The most common global measure is the area under the curve (AUC) of the ROC plot. The area under the ROC curve is a measure of the probability that the perceived measurement will allow correct identification of a condition. By convention, this area is always ≥0.5. Values range between 1.0 (perfect separation of the test values of the two groups) and 0.5 (no apparent distributional difference between the two groups of test values). The area does not only depend on a portion of the plot such as the point closest to the diagonal or the sensitivity at 90% specificity, but on the entire plot. This is a quantitative, descriptive expression of how close the ROC plot is to the perfect one (area=1.0).

Methods & Results

Patients

Seventy-eight age and sex-matched participants were recruited in the US by Discovery Life Sciences (DLS), 1236 Los Osos Valley Rd, Suite T, Los Osos, Calif. 93402 USA and PrecisionMed, 132 N. Acacia Ave, Solana Beach, Calif. 92075, USA. The participants comprised a PTSD cohort (N=39) and a non-PTSD cohort (N=39). Venous blood samples and a detailed clinical history were collected for each study participant. The study conformed to all Data Use Agreements (DUA). Socio-demographic and clinical factors were collected from each participant and included; age, gender, prescribed medications, and comorbidities e.g. depression, anxiety, panic disorder, diabetes, and hypertension. Medications being prescribed to patients at the time of the blood draw were: Abilify, Adalat, Atarax, Ativan, Baclyfen, Benzarpil, Buspirone HCl, Celexa, Clozapine, Crestor, Cyclobenzaprine, Cymbalta, Depakote, Doxycycline, Effexor, Eliquis, Fetzima, Gabapentin, Hydrochlorothiazide, Imitrex, Inderal, Irbesartan, Janumet, Klonopin, Lamictal, Lasix, Latuda, Lexapro, Lovastatin, Maxalt, Metformin, Minocycline, Mobic, Nexium, Norco, Paroxetine, Plaquenil, Prozac, Prazosin, Saphris, Seroquel, Trazadone, Viibryd, Xanax, Zoloft and Zyrtec.

A follow-up study (N=84) measured the level of GFAP in individuals without PTSD. Medications being prescribed to patients at the time of the blood draw were: Atorvastatin, Azathioprine, Cetirizine, Chlorphenamine, Cortisone, Desmopressin, Diazepam, Diclofex, Effexor, Femoston, Flixonase, Fosamax, Hydrocortisone, Lansoprazole, Levothyroxine, Lisinopril, Loperamide, Metformin, Methotrexate, Midazolam, Mycophenolate, Omeprazole, Perindopril, Prednisone, Premarin, Propanolol, Propecia, Prozac, Ramipril, Rosuvastatin, Sertraline, Simvastatin, Spiriva, Symbicort, Thyroxine, Warfarin.

Sampling and Laboratory Methods

Scientists, blinded to participant data, completed analyses of biomarkers at Randox Clinical Laboratory Services (RCLS) (Antrim, UK) using cytokine arrays (Randox Laboratories Ltd, Crumlin, UK) for the following proteins: Cytokine I Array: Interleukin-1α, −1β, −2, −4, −6, −8, −10, VEGF, EGF, TNFα, IFNγ and MCP-1; Metabolic Array I: Ferritin, insulin, leptin, plasminogen activator inhibitor-1 (PAI-1), and resistin; Metabolic Array II: C-reactive protein (CRP), adiponectin and cystatin C; Cerebral Array I: Brain-derived neurotropic factor (BDNF), glial fibrillary acidic protein (GFAP), and heart-type fatty acid binding protein (H-FABP); Cerebral Array II: D-dimer, neuron specific enolase (NSE), neutrophil gelatinase-associated lipocalin (NGAL), and soluble tumour necrosis factor receptor I (sTNFR1). Arrays were run on Evidence Investigator© analyzers according to manufacturer's instructions (Randox Laboratories Ltd, Crumlin, UK). Cholesterol (total), HDL and LDL cholesterol were analyzed on Randox RX Series analyzers (RCLS, Antrim, UK). Human tissue-type plasminogen activator (tPA) and human type 1 plasminogen activator inhibitor PAI-1/tPA complex ELISAs were obtained from AssayPro, 3400 Harry S. Truman Blvd, St. Charles, Mo. 63301. Assays were completed according to manufacturer's instructions. The limits of detection (LOD) for the biomarkers under investigation were as follows: Cytokine I—IL-2 2.97 pg/ml; IL-4 2.12 pg/ml; IL-6 0.12 pg/ml, IL-8 0.36 pg/ml; VEGF 3.24 pg/ml; IFNγ 0.44 pg/ml; TNFα 0.59 pg/ml; IL1α 0.19 pg/ml; MCP1 3.53 pg/ml; EGF 1.04 pg/ml; IL-10 0.37 pg/ml; IL-1β 0.26 pg/ml; Metabolic Array I—Ferritin 3.27 ng/ml, insulin 2.32 pIU/ml, leptin 1.10 ng/ml, PAI-1 2.34 ng/ml, and resistin 1.06 ng/ml; Metabolic Array II—CRP 0.69 mg/l, adiponectin 164 ng/ml, and cystatin C 60 ng/ml; Cerebral Array I—BDNF 0.59 pg/ml, and GFAP 0.18 ng/ml; Cerebral Array II—D-dimer 2.1 ng/ml, NSE 0.26 ng/ml, NGAL 17.8 ng/ml, and STNFRI 0.24 ng/ml. Direct HDL—cholesterol (HDL) 0.189 mmol/l (7.30 mg/dl), direct LDL—cholesterol (LDL) 0.189 mmol/l (7.30 mg/dl), cholesterol 0.865 mmol/l (33.4 mg/dl). AssayPro ELISA—Human tPA ELISA—0.013 ng/ml and human PAI-1tPA complex ELISA—0.05 ng/ml. Thirty-two biomarkers in serum were investigated. Biomarker values below the LOD were assigned 90% the LOD value.

Statistical Analysis & Results

No gender effects were observed in either study.

Main study. There was no significant difference in BMI, pulse rate or systolic blood pressure between the control (non-PTSD) and PTSD groups. Diastolic blood pressure for the PTSD group was greater (p=0.004). PTSD individuals presented with significantly more comorbidities than the control group e.g. panic disorder, depression, and anxiety. PTSD individuals were prescribed significantly more medications than the control group (2.8±2.4 vs. 0.7±1.2, p<0.001, respectively). The GFAP levels within PTSD patients (N=39) and within non-PTSD patients (N=39) with/without depression and medication were analyzed using one-way anova with Tukey's comparison; all other analyses were by way of t-test. Significant levels of P<0.05 and P<0.10 were used. All data was analyzed using Graphpad Prism following log₁₀ transformation where applicable.

The two age-matched cohorts (gender did not influence GFAP levels) analyzed were

1. PTSD patients/individuals: with depression on medication (D+M), without depression on medication (no D+M) and without depression and not on medication (no D+no M)

2. Non-PTSD patients/individuals (Control group): with depression on medication (D+M), without depression on medication (no D+M) and healthy patients (H).

Results are summarized in Table 1.

TABLE 1
GFAP level statistical analysis (one-way anova) for depression and medication
within PTSD patient cohort and non-PTSD patient cohort (Control Group)

| PTSD patient cohort | | Non-PTSD patient cohort | |
|---|---|---|---|
| P-value | | P-value | |
| (D + M) vs (no D + M) | ns | (D + M) vs (no D + M) | ns |
| (D + M) vs (no D + no M) | P < 0.05 | (D + M) vs (H) | P < 0.001 |
| (no D + M) vs (no D + no M) | P < 0.05 | (no D + M) v (H) | P < 0.001 |

| | |
|---|---|
| Overall P-value = 0.0185 | Overall P-value = <0.0001 |

D = depression,
M = medication,
H = healthy (no medication, no disease)

In the non-PTSD cohort, GFAP levels in the serum of both depressed and non-depressed individuals on medication were significantly increased compared to healthy individuals (P<0.001). In the PTSD cohort, medication is also shown to significantly increase GFAP levels in serum of both depressed and non-depressed individuals on medication compared to healthy individuals (P<0.05). ROC curve analysis supported these findings (Table 2). PTSD individuals who were neither on medication nor depressed and healthy individuals had similar levels of GFAP (0.26 vs 0.28 ng/ml; FIG. 1). In both the PTSD and non-PTSD cohorts depressed individuals on medication had greater absolute amounts of blood GFAP than non-depressed individuals on medication.

TABLE 2
ROC curve analyses of GFAP levels for depression
and medication within PTSD patient cohort and
non-PTSD patient cohort (Control Group).

| | % Sensitivity | % Specificity | AUC |
|---|---|---|---|
| PTSD patient cohort | | | |
| (D + M) vs (no D + M) | 64 | 50 | 0.5503 |
| (D + M) vs (no D + no M) | 67 | 91 | 0.9242 |
| (no D + M) vs (no D + no M) | 67 | 93 | 0.9167 |
| Non-PTSD patient cohort | | | |
| (D + M) vs (no D + M) | 50 | 100 | 0.8750 |
| (D + M) vs (H) | 96 | 100 | 0.9904 |
| (no D + M) vs (H) | 88 | 100 | 0.9423 |

D = depression,
M = medication

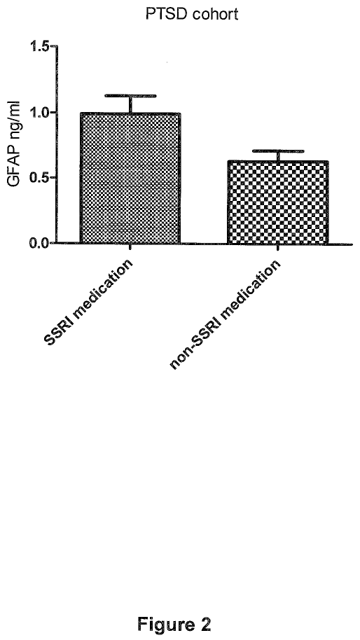

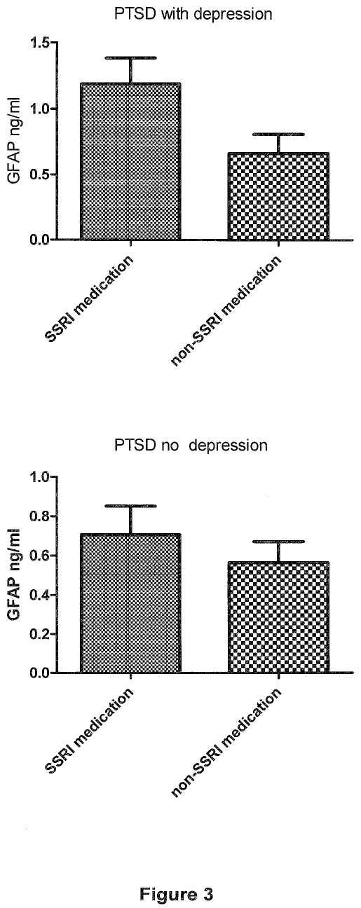

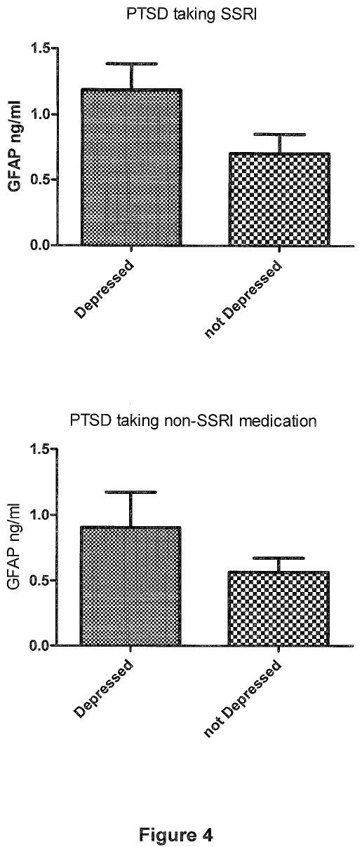

The effect of medication was further analyzed by medication type using the PTSD cohort. Individuals on medication taking a selective-serotonin reuptake inhibitor (SSRI) had significantly higher GFAP levels than individuals on medication not taking a SSRI (P<0.05; FIG. 2). This analysis was further supported by a comparison of GFAP levels of depressed individuals taking SSRI medication versus depressed individuals on non-SSRI medication (P=<0.05, FIG. 3 top). In non-depressed PTSD individuals, absolute levels of GFAP were greater in SSRI medicated individuals than non-medicated individuals (P>0.05, FIG. 3 bottom). GFAP levels in depressed individuals were increased compared to non-depressed individuals in both SSRI (P<0.010) and non-SSRI medicated groups (FIG. 4).

Within the non-PTSD cohort, individuals taking non-prescribed, non-steroidal anti-inflammatory medication (NSAIDs) had no detectable levels of GFAP. Individuals taking SSRI medication had higher levels of GFAP than Individuals taking non-SSRI medication (1.01 ng/ml vs 0.85 ng/ml). To determine if there was a possible effect caused by the number of drugs being taken per individual on the level of GFAP, a correlation analysis of GFAP levels vs number of medications taken was performed; there was no significant correlation (r=0.212, P>0.05). The results demonstrate that GFAP levels are increased in the blood of both PTSD and healthy individuals who are taking prescribed medication, individuals taking SSRI medication, and individuals who are depressed.

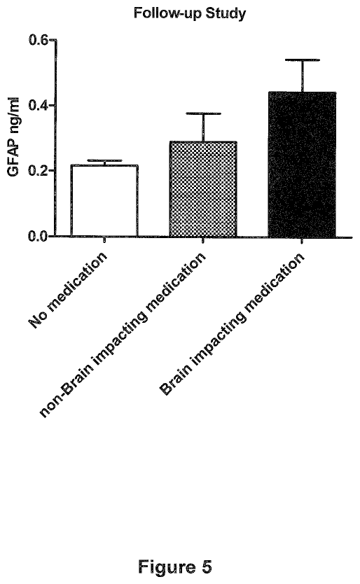

Follow-up Study. The follow-up study, comparing individuals on no medication, non-brain impacting medication and brain impacting medication was analyzed using one-way anova with Tukey's comparison. As previously described, brain impacting medication includes drugs interacting with cell receptors located in the brain (both target and off-target drugs) or interfere with brain physiological pathways, including neurotransmission processes. The follow-up study results (Table 3), confirm that medication impacting the brain increases GFAP levels (one-way anova P=0.0113). individuals taking medication active against receptors located in the brain or brain physiological pathways had significantly greater levels of serum GFAP than individuals not taking medication (P<0.01). Also, individuals on other medication (central bas graph of FIG. 5) had greater average absolute levels of GFAP than patients not taking medication. Re-analysis of the data omitting five patients with depression did not affect the result.

TABLE 3
Comparison of GFAP levels in the serum of individuals
not taking medication (N = 48) vs individuals
taking medication (N = 36)

| | Mean GFAP levels (ng/ml) |
|---|---|
| Not taking medication (N = 48) | 0.21 |
| Taking non-brain impacting | 0.29 |
| medication (N = 20) | |
| Taking brain impacting | 0.44 |
| medication (N = 16) | |

A possible interpretation of these findings is that astrocyte cells (or other GFAP-containing cells) are breaking down and leaking into the peripheral circulation, driven by medication and depression. As with most drugs, the systemic response to medication can vary on an individual basis; some individuals may experience more severe brain cellular impacts and damage than others, while others will be unaffected, and the level of GFAP in the blood will vary accordingly. An advantage of the current invention is that by identifying individuals whose GFAP blood levels increase following medication, especially medication used for treating psychotic disorders such as depression which is believed to disrupt the brain's neurotransmission systems, a clinician can make a more informed cost-benefit evaluation on an individual basis and potentially prescribe a different drug to manage the patient's condition. A point of care test would readily enable the clinician (hospital-based or general-practice based) to take a patient's blood sample before and during drug treatment, measure the amount of GFAP, and respond accordingly. Alternatively, the patient could readily and rapidly self-monitor GFAP blood levels (e.g. using blood from a finger-prick sample) during drug treatment at home using a suitable medical device. The impact of medication on cellular damage as assessed by GFAP concentrations could also be readily applied using in vitro cellular models and methods which would be of great benefit during the drug development process. A further advantage derived from the current findings is the ability to support a clinician's diagnosis of depression using an objective measure for depression i.e. GFAP concentrations. Given the difficult nature of diagnosing mental-health-related illnesses and the social stigma that often accompanies these conditions, this discovery could significantly benefit the patient.

### REFERENCES

1. American Psychiatric Association. Diagnostic and Statistical Manual of Mental Disorders [Internet]. American Psychiatric Association; 2013 [cited 2019 Jul. 4]. Available from: https://psychiatryonline.org/doi/book/10.1176/appi.books.9780890425596
2. Moeton M. et al. (2016), Cellular and Molecular Life Sciences, 73: 4101-4120.
3. Mayer C. A. et al. (2013), PLoS ONE, 8(4): e62101.

1. A method of assessing the cellular toxicity of a drug comprising administering a drug to an individual or to an in vitro cell line or cell model, measuring the amount of GFAP in an in vitro sample taken from the individual or in the in vitro cell line or cell model and comparing the amount of GFAP measured to a control measurement in which a GFAP measurement taken from the in vitro sample or in vitro cell line or cell model which is greater than the control measurement is indicative of cellular toxicity.

2. The method of claim 2 in which the control measurement is a GFAP level measurement obtained from the in vitro sample or in vitro cell line or cell model prior to administering the drug.

3. The method of claim 1 in which the drug is for use in treating a brain-related pathology.

4. The method of claim 1 in which the drug affects the neurotransmission pathway in the brain.

5. The method of claim 4 in which the drug is a selective-serotonin reuptake inhibitor, a selective-dopamine reuptake inhibitor, selective-norepinephrine reuptake inhibitor, a serotonin receptor agonist or antagonist, a dopamine receptor agonist or antagonist, a GABA receptor agonist or antagonist, an adrenergic receptor agonist or antagonist, a glutamate receptor agonist or antagonist, a glycine receptor agonist or antagonist or an opioid receptor agonist or antagonist.

6. The method of claim 4 in which the drug is a selective-serotonin reuptake inhibitor, a selective-dopamine reuptake inhibitor, selective-norepinephrine reuptake inhibitor, a serotonin receptor agonist or antagonist or a dopamine receptor agonist or antagonist.

7. The method of claim 4 in which the drug is a selective-serotonin reuptake inhibitor.

8. The method of claim 3 in which the brain-related pathology is depression, anxiety, stress, panic disorder, pain, epilepsy, dementia, suicidal ideation, Alzheimer's disease or Parkinson's disease.

9. The method of claim 3 in which the brain-related pathology is depression.

10. A method of supporting a diagnosis of depression in an individual comprising taking a sample from an individual, measuring the amount of GFAP in the sample and comparing the amount of GFAP measured to a control measurement in which a GFAP measurement taken from the sample which is greater than the control measurement is supportive of the individual being depressed.

11. The method of claim 10 in which the control measurement is a GFAP level measurement obtained from the individual prior to administering the drug and preferably at a time point when the individual is not subject to depression.

12. The method of claim 10 in which the sample is blood, serum or plasma.

13. A method of determining drug-induced cellular toxicity comprising measuring GFAP in a cellular sample.

14. The method of claim 13 in which the drug binds to cellular receptors located in the brain or interrupts physiological pathways in the brain.

15. The method of claim 14 in which the drug is a selective-serotonin reuptake inhibitor, a selective-dopamine reuptake inhibitor, selective-norepinephrine reuptake inhibitor, a serotonin receptor agonist or antagonist or a dopamine receptor agonist or antagonist.

16. The method of claim 14 in which the drug is a selective-serotonin reuptake inhibitor.

17. A method of determining depression in a subject comprising measuring GFAP in a biological sample obtained from the subject.

18. The method of claim 13 in which GFAP is derived from blood, serum or plasma.

19. The method of claim 17 in which GFAP is derived from blood, serum or plasma.
